(19) United States
(12) Patent Application Publication
(10) Pub. No.: US 2005/0209544 A1
(43) Pub. Date: Sep. 22, 2005

(54) Hand protector used in recuperation process

(76) Inventor: Chin-Tsun Lee, Yongkang City (TW)

Correspondence Address: CHIN-TSUN LEE, P.O. Box 90, Tainan City, 704, TW

(21) Appl. No.: 10/805,556

(22) Filed: Mar. 18, 2004

(51) Int. Cl. A61F 5/00

(52) U.S. Cl. 602/21

(57) ABSTRACT

A hand protector used in recuperation process is made of a plastic material, including a palm-supporting member, and a finger-supporting member pivotally connected with the palm-supporting member. The hand protector can further be added with a wrist-supporting member connected rearward with the palm-supporting member. Thus, a user can use a hand protector of a proper length according to different sections of the hand to be recuperated, supporting the different sections of the hand in the stable normal condition, and with the pivotal connection enabling the joints of the hand to swing freely for stimulating smooth circulation of blood so as to prevent the hand from deforming or suffering from atrophy to obtain the best effect of recuperation.

### BACKGROUND OF THE INVENTION

1. Field of the Invention

This invention relates to a hand protector used in recuperation process, particularly to one possible to adjust its length for the different sections of the hand to be recuperated, holding the sections of the hand in a natural normal condition, with a connecting means connecting pivotally the different sections of the hand, so the joints of the hand may move up and down to exercise, keeping smooth circulation of blood so as to prevent a hand from deforming or shriveling and obtain the best effect in hand recuperating process.

2. Description of the Prior Art

A conventional hand protector used in recuperation process shown in FIG. 1 includes a plastic plate 1 made of resin mixed with hardener, which is softened by immersing it in hot water of 40° C.-50° C., and then cut off a part corresponding to the opening between the thumb and the index finger, and then is kneaded manually into a general shape of a hand, and then trimming its circumferential line neatly. Then a user wears it for making recuperation process to a hand.

However, this conventional hand protector has to be made by kneading the considerably hot plastic plate 1 into a proper shape, in addition to its high cost of the material, and necessary trimming of its circumferential line to waste not a little material. Further, should the temperature of the plastic plate 1 becomes lower than 40° C., it might have to be heated once again for kneading, quite troublesome. Besides, a worker is liable to be scalded in treating the hot material, taking time and causing potential risk to result in high cost.

Another conventional hand protector 2 shown in FIG. 2 includes plural binding members 20 to be bound on different sections of a hand and also connected by a steel line 21 in place, and an elastic member 22 provided between the two ends of the steel line 21 so there is an elasticity to pull back the hand in recuperating process after the steel line is pulled.

Meanwhile, the second conventional hand protector 2 cannot hold the palm and the fingers stably, so the thumb and the other fingers may deform or shrivel owing to muscular atrophy or dystrophy.

### SUMMARY OF THE INVENTION

This invention has a purpose to offer a hand protector used in recuperation process, which can hold the hand in a normal condition of smooth blood circulation and let various joints of the hand bend and exercise to obtain the best effect in hand recuperation process.

A first feature of the invention is a finger supporting member and a palm supporting member included in a hand protector made of a plastic material, and an opening bottom wall formed in the hand protector to correspond to the opening between the thumb and the index finger, and a thumb-supporting member. The palm supporting member and the finger-supporting member are pivotally connected to permit the joints of the hand bend freely in hand recuperating process.

A second feature of the invention is the palm supporting member, the opening bottom wall, the thumb supporting member, and the finger supporting member respectively for the palm, the opening between the thumb and the index finger, the thumb and the other four fingers to rest on for convenience of hand recuperating.

### BRIEF DESCRIPTION OF DRAWINGS

This invention will be better understood by referring to the accompanying drawings, wherein:

FIG. 1 is a flowing chart of a conventional hand protector used in recuperation process;

FIG. 2 is a perspective view of another conventional hand protector worn on a hand for recuperation process;

FIG. 3 is an exploded perspective view of a first embodiment of a hand protector used in recuperation process in the present invention;

FIG. 4 is a perspective view of the first embodiment of a hand protector used in recuperation process in the present invention;

FIG. 5 is a perspective view of the first embodiment of a hand protector with a different connecting structure from that shown in FIG. 4 for a palm-supporting member with a finger-supporting member in the is present invention;

FIG. 6 is a second embodiment of a hand protector used in recuperation process in the present invention; and, FIG. 7 is a third embodiment of a hand protector used in recuperation process in the present invention.

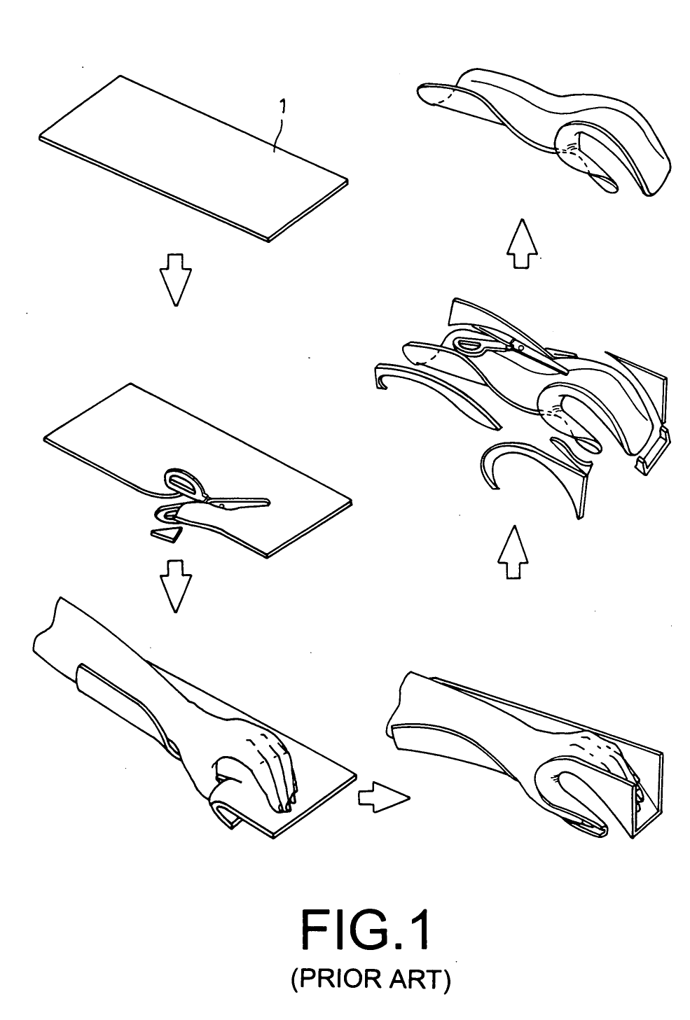

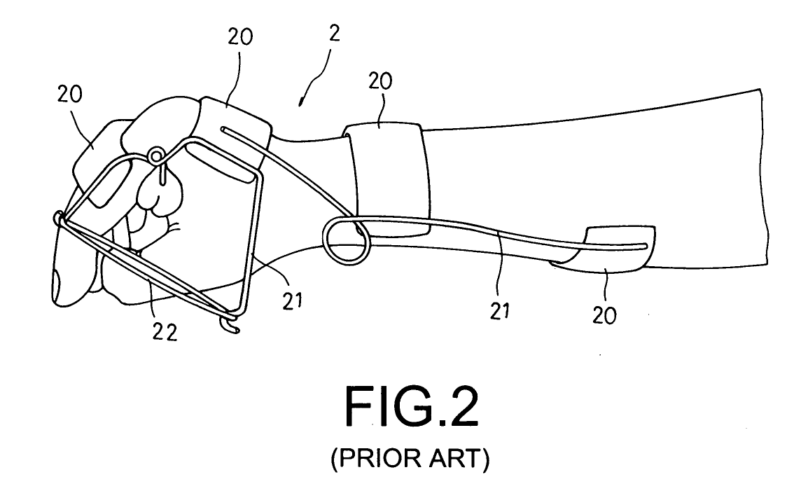

### DETAILED DESCRIPTION OF THE PREFERRED EMBODIMENT

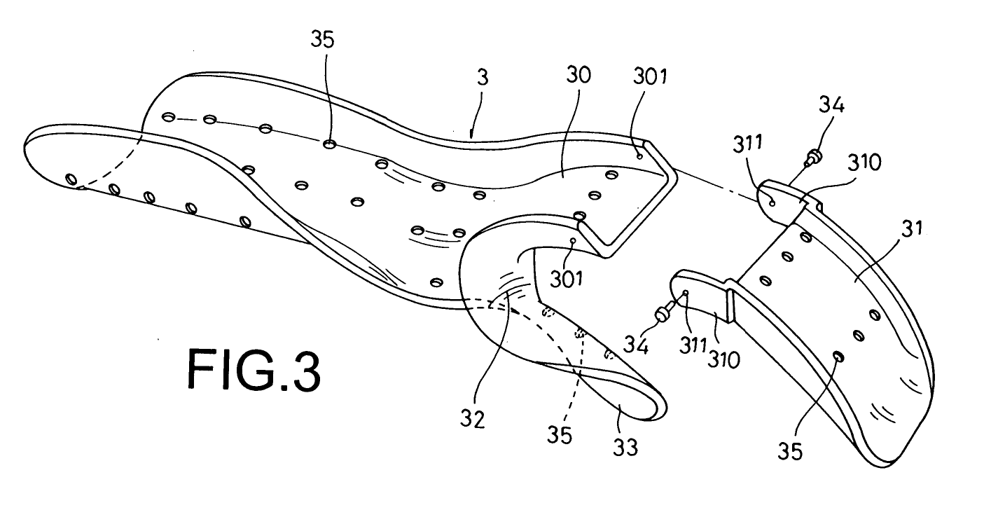

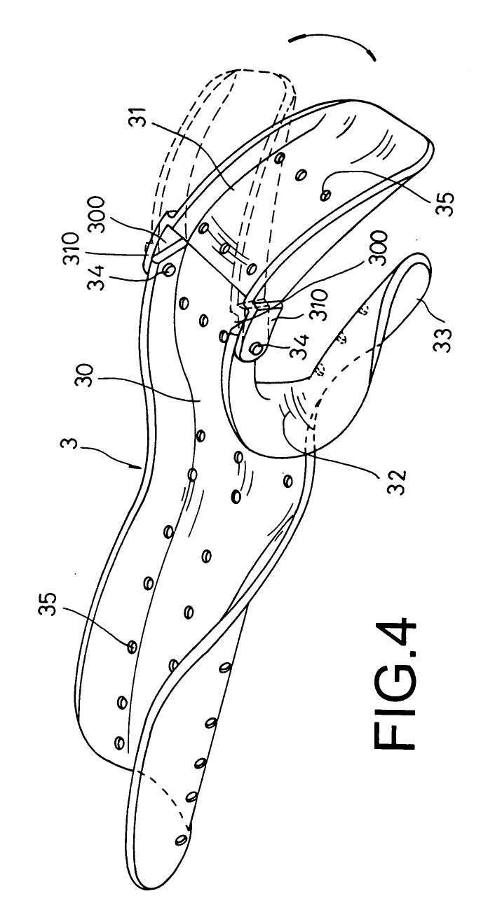

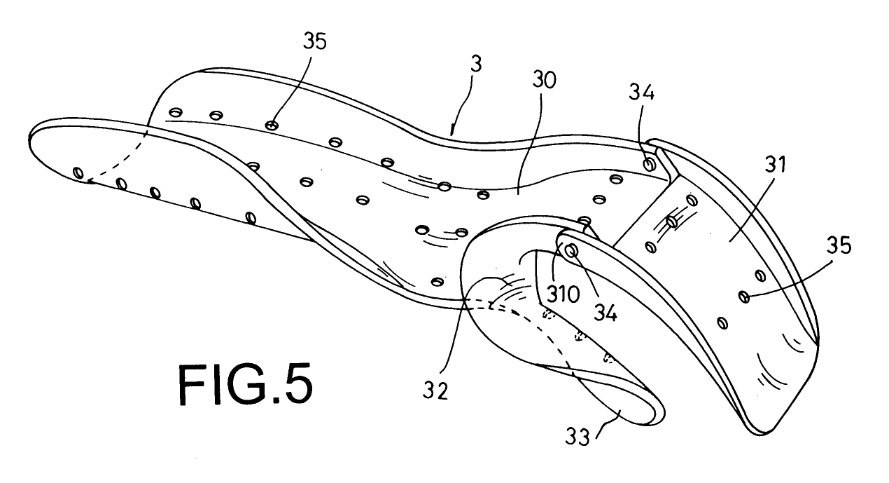

A first preferred embodiment of a hand protector 3 used in recuperation process, as shown in FIGS. 3 and 4, includes a palm-supporting member 30 and a finger-supporting member 31 both made of a plastic material.

The palm-supporting member 30 has an opening bottom wall 32 for an opening between the thumb and the index finger to fit with, a thumb-supporting member 33 formed under said opening bottom wall 32 for the thumb to rest against. Further, the palm-supporting member 30 has plural air holes 35 spaced apart near the front end.

The finger-supporting member 31 has two ears 310 with a hole 311 formed respectively from the rear ends of the two sidewalls and a little bending outward, with the distance between the two ears 310 being a little larger than the distance between the two sidewalls of the finger-supporting member 31 and the palm-supporting member 30. Then two connect pins 34 fits in the hole 311 and 301 to connect pivotally the finger-supporting member with the palm-supporting member 30. So there is a small gap 300 formed respectively between the two sidewalls of the palm-supporting member 31 and the ears 310 of the finger-supporting member 31. Further the finger-supporting member 31 has plural air holes 35 spaced apart in the intermediate portion. Then the two ears 310 of the finger-supporting member 31 are pivotally connected with the front ends of he two sidewalls of the palm-supporting member 30.

FIG. 5 shows a different connecting structure from that shown in FIG. 4 for the palm-supporting member 30 with the finger-supporting member 31, with the ears 310 of the sidewalls of the finger-supporting member 31 extending straight from the rear ends of the two sidewalls of the finger-supporting member 31.

In using the first embodiment of the hand protector 3, it can be bound by means of an adhesive tape or bandage properly on the hand of a user, and the user can swing the finger-supporting member 31 up and down by the pivotal connection of the finger-supporting member 31 with the palm-supporting member 30, stimulating smooth circulation of blood to prevent the hand from deforming, shriveling, etc.

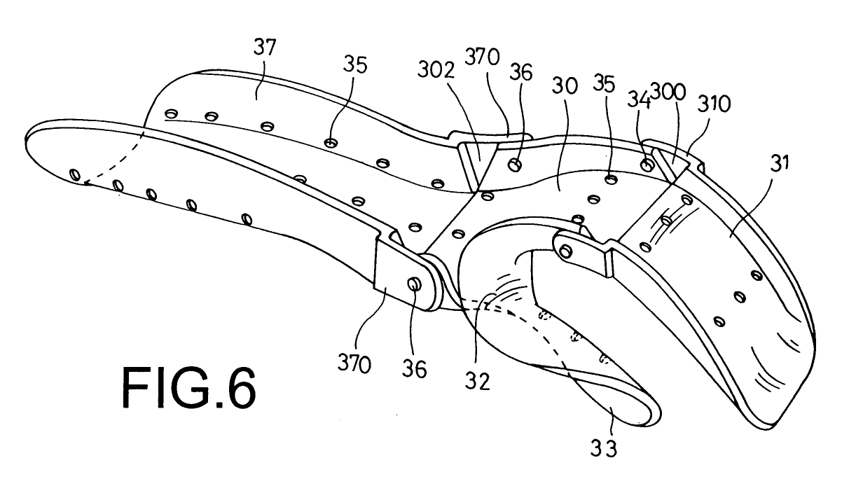

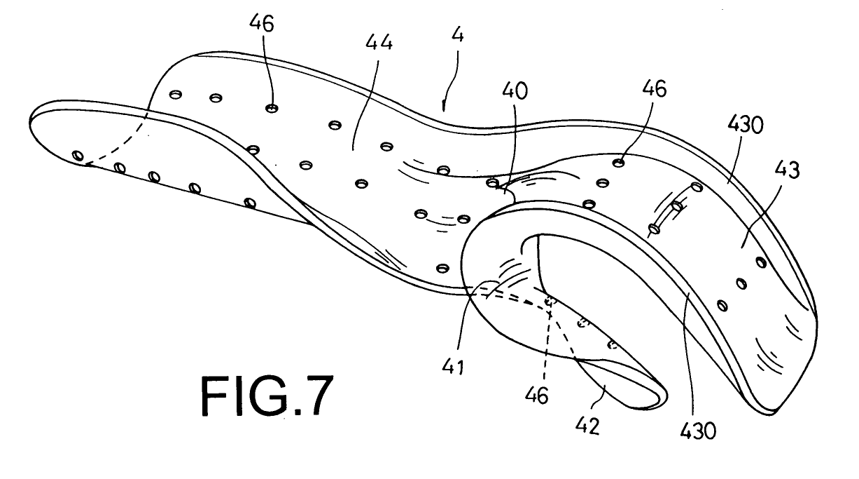

Next, FIG. 6 shows a second embodiment of a hand protector used in recuperation process, which includes a palm-supporting member 30, a finger-supporting member 31, and a wrist-supporting member 37 having two ears 370 at two sides of the front end. The wrist-supporting member 37 is pivotally connected with the palm-supporting member 30 by connect pins 36. The finger-supporting member 31 has two ears 310 at two sides of the rear end, and pivotally connected with the two sides of the front end of the palm-supporting member 30 in the same way as the first embodiment. Further, the palm and the finger and the wrist-supporting member 30, 31 and 37 all have plural air holes 35.

Then the second embodiment can be used in almost the same way as the first embodiment in recuperation process.

Moreover, FIG. 7 shows a third embodiment of a hand protector 4 used in recuperation process, which is formed integral, having a palm-supporting member 40, an opening bottom wall 41, a thumb-supporting member 42 and a finger-supporting member 43 and a wrist-supporting member 44 all not pivotally connected with one another. The opening bottom wall 41 supports the opening between the thumb and the index finger, and the thumb-supporting member 42 is formed under the opening bottom wall 41 and has a curved cross-section for the thumb to rest against, and the finger-supporting member 43 is located in front of the palm-supporting member 40 and shaped curved a little to suit the other four fingers. The finger-supporting member 43 has two sidewalls 430 lengthwise for restricting the other four fingers. The wrist-supporting member 44 is located behind the palm-contacting member 40, having two lengthwise sides curved up. Further, an arm-supporting member can be added to extend rearward from the rear end of the wrist-supporting member 44, if needed. There are plural air holes 46 in the whole hand protector 4.

In using the third embodiment of the hand protector 4, the opening between the thumb and the index finger is rested against the opening bottom wall 41, with the thumb resting against the thumb-supporting member 42, with the other four fingers resting against the finger-supporting member 43 and restricted by the two sidewalls 430 stably. Then a hand of a user is supported by the hand protector 4, keeping the fingers and the palm in a natural normal condition, preventing the hand from deforming, shriveling, etc. owing to muscular atrophy to obtain the best effect of hand recuperation.

The invention has the following advantages, as can be understood from the aforesaid description.

1. It can be used for different sections of a hand for recuperation, by using it with different length, keeping smooth circulation of blood, preventing the hand from deforming, shriveling, etc., and obtaining the best effect of recuperation.

2. It can save time and material in manufacturing, without wasting material and thus reducing its cost.

While the preferred embodiments have been described above, it will be recognized and understood that various modifications may be made therein and the appended claims are intended to cover all such modifications that may fall within the spirit and scope of the invention.

1. A hand protector used in recuperation process, said hand protector made of a plastic material and comprising a finger-supporting member, a palm-supporting member, said palm-supporting member having an opening bottom wall for an opening between the thumb and the index finger to rest against, a thumb-supporting member formed under said opening bottom wall, said palm-supporting member and said finger-supporting member pivotally connected at least with one connecting pin so that said finger-supporting member is swingable relative to said palm-supporting member.

2. The hand protector used in recuperation process as claimed in claim 1, wherein said palm-supporting member having two sidewalls at a front end, said two sidewalls respectively having a sidewise hole, said finger-supporting member having an ear respectively at two sidewalls of a rear end, each said ear having a sidewise hole, two connect pins respectively extending in said sidewise holes of said palm and said finger-contacting member to pivotally connect said palm and said finger-supporting member, a wrist-supporting member formed behind said palm-supporting member, said wrist-supporting member connected pivotally with said palm-supporting member at least by a connect pin, said hand protector having plural air holes.

3. A hand protector used in recuperation process, said hand protector made of a plastic material and comprising a palm-supporting member, an opening bottom wall, a thumb-supporting member and a finger-supporting member; said palm-supporting member for a palm of a hand to rest against, said opening bottom wall curve-shaped for an opening between the thumb and the index finger to fit with, said thumb-supporting member located under said opening bottom wall for the thumb to rest against, said finger-supporting member located in front of said palm-supporting member for the other four fingers to rest ageist, said hand protector shaped integral and inflexible.

4. The hand protector used in recuperation process as claimed in claim 3, wherein a wrist-supporting member is provided to extend rearward from the rear end of said palm-supporting member, and said hand protector has plural air holes.
